(19) United States
(12) Patent Application Publication
(10) Pub. No.: US 2016/0331249 A1
(43) Pub. Date: Nov. 17, 2016

(54) VITAL SIGN MEASUREMENT SYSTEM AND VITAL SIGN MEASUREMENT METHOD THEREOF

(71) Applicant: Cheng Uei Precision Industry Co., Ltd., New Taipei City (TW)

(72) Inventors: James Cheng Lee, New Taipei City (TW); Kuo Yang Wu, New Taipei City (TW); Wen Bing Hsu, New Taipei City (TW); Hsiang Ling Chung, New Taipei City (TW)

(21) Appl. No.: 14/712,961

(22) Filed: May 15, 2015

(51) Int. Cl. A61B 5/0205; A61B 5/00; A61B 5/053

(52) U.S. Cl. CPC A61B 5/02055; A61B 5/053; A61B 5/7203; A61B 5/021

(57) ABSTRACT

A vital sign measurement system includes an excitation signal source, a vital sign sensor, an environmental sensor, an analog front-end sensor electrically connected with the vital sign sensor, and a processor module electrically connected with the excitation signal source, the environmental sensor and the analog front-end sensor. The excitation signal source generates excitation signals. The excitation signals are transmitted to a human body, and then the excitation signals are reflected to generate sensing signals. The vital sign sensor receives the sensing signals, and the sensing signals are converted into a plurality of analog synthetic signals. The environmental sensor receives environmental light. The analog front-end sensor receives and amplifies the analog synthetic signals. The processor module calculates an intensity of the environmental light. And the processor module sends an adjustment instruction to the analog front-end sensor to adjust a sampling frequency of the analog front-end sensor.

### BACKGROUND OF THE INVENTION

1. Field of the Invention

The present invention generally relates to a vital sign measurement system, and more particularly to a vital sign measurement system which is capable of improving accuracies of measuring vital signs, and a vital sign measurement method thereof.

2. The Related Art

Nowadays, a conventional vital sign measurement system is widely used for measuring vital signs. The conventional vital sign measurement system based on a light sensor generally includes a light source and a light sensor. Light emitted by the light source irradiates on skin of a human body and is reflected, and then the light sensor senses changes of reflected light in a period of time to get Photoplethysmography (PPG) signals so as to detect the changes of the vital signs.

However, as for the conventional vital sign measurement system based on the light sensor, the PPG signals are easily disturbed on account of interferences of ambient light and ultraviolet rays. The conventional vital sign measurement system based on a bio-impedance sensor generally includes a bio-impedance sensor. As for the conventional vital sign measurement system based on the bio-impedance sensor, bio-electrical signals sensed by the bio-impedance sensor are extremely weak, and are hardly distinguished under the interferences of the ambient light and the ultraviolet rays. As a result, accuracies of measuring the vital signs are affected.

In view of this, it's essential to provide an innovative vital sign measurement system which is capable of improving the accuracies of measuring the vital signs, and a vital sign measurement method of the innovative vital sign measurement system.

### SUMMARY OF THE INVENTION

An object of the present invention is to provide a vital sign measurement system and a vital sign measurement method thereof. The vital sign measurement system includes an excitation signal source, a vital sign sensor, an environmental sensor, an analog front-end sensor and a processor module. The excitation signal source generates excitation signals. The excitation signals are transmitted to skin of a human body, and then the excitation signals are reflected by the human body to generate sensing signals. The vital sign sensor receives the sensing signals reflected by the human body, and the sensing signals are converted into a plurality of analog synthetic signals by the vital sign sensor. The environmental sensor receives environmental light of external ambient where the vital sign measurement system is located. And the environmental light is converted into a plurality of ambient digital indices by the environmental sensor. The analog front-end sensor is electrically connected with the vital sign sensor. The analog front-end sensor receives and amplifies the analog synthetic signals of the vital sign sensor. And the analog synthetic signals are converted into biological digital signals. The processor module is electrically connected with the excitation signal source, the environmental sensor and the analog front-end sensor. The processor module calculates an intensity of the environmental light of the external ambient on the basis of the ambient digital indices. And the processor module sends an adjustment instruction to the analog front-end sensor to adjust a sampling frequency of the analog front-end sensor according as the environmental sensor senses an intensity change of the environmental light of the external ambient. The processor module proceeds an estimation and a measurement of vital signs according as the processor module receives the biological digital signals converted by the analog front-end sensor of which the sampling frequency is adjusted.

Another object of the present invention is to provide a vital sign measurement method. Steps of the vital sign measurement method of the vital sign measurement system which includes an excitation signal source, a vital sign sensor, an environmental sensor, an analog front-end sensor and a processor module are described hereinafter. The processor module controls the excitation signal source to generate excitation signals. The excitation signals are transmitted to skin of the human body, and then the excitation signals are reflected by the human body to generate sensing signals. The vital sign sensor receives the sensing signals. And the sensing signals are converted into a plurality of analog synthetic signals by the vital sign sensor. The environmental sensor receives environmental light of external ambient where the vital sign measurement system is located. And the environmental light is converted into a plurality of ambient digital indices. The analog front-end sensor receives and amplifies the analog synthetic signals of the vital sign sensor. And the analog front-end sensor converts the analog synthetic signals into biological digital signals. The processor module calculates an intensity of the environmental light of the external ambient on the basis of the ambient digital indices. And the processor module sends an adjustment instruction to the analog front-end sensor to adjust a sampling frequency of the analog front-end sensor. The analog front-end sensor of which the sampling frequency is adjusted amplifies the analog synthetic signals and converts the analog synthetic signals into the biological digital signals. The processor module proceeds an estimation and a measurement of vital signs according as the processor module receives the biological digital signals converted by the analog front-end sensor of which the sampling frequency is adjusted.

As described above, the vital sign measurement system adjusts the sampling frequency of the analog front-end sensor according as the environmental sensor senses the intensity change of the external ambient where the vital sign measurement system is located, when the intensity of the environmental light becomes larger, the sampling frequency of the analog front-end sensor is higher, when the intensity of the environmental light becomes smaller, the sampling frequency of the analog front-end sensor is lower, so that measured values of the vital signs affected by the environmental light are balanced. As a result, accuracies of the vital sign measurement system measuring the vital signs are assured.

### BRIEF DESCRIPTION OF THE DRAWINGS

This present invention will be apparent to those skilled in the art by reading the following description, with reference to the attached drawings, in which:

FIG. 1 is a functional block diagram of a vital sign measurement system in accordance with the present invention;

FIG. 2 is a functional block diagram of the vital sign measurement system in accordance with a first embodiment of the present invention;

FIG. 3 is a functional block diagram of the vital sign measurement system in accordance with a second embodiment of the present invention;

FIG. 4 is a functional block diagram of a vital sign measurement system in accordance with a third embodiment of the present invention;

FIG. 5 is a functional block diagram of a vital sign measurement system in accordance with a fourth embodiment of the present invention; and

FIG. 6 is a flow chart of a vital sign measurement method of the vital sign measurement system of FIG. 1.

### DETAILED DESCRIPTION OF THE EMBODIMENT

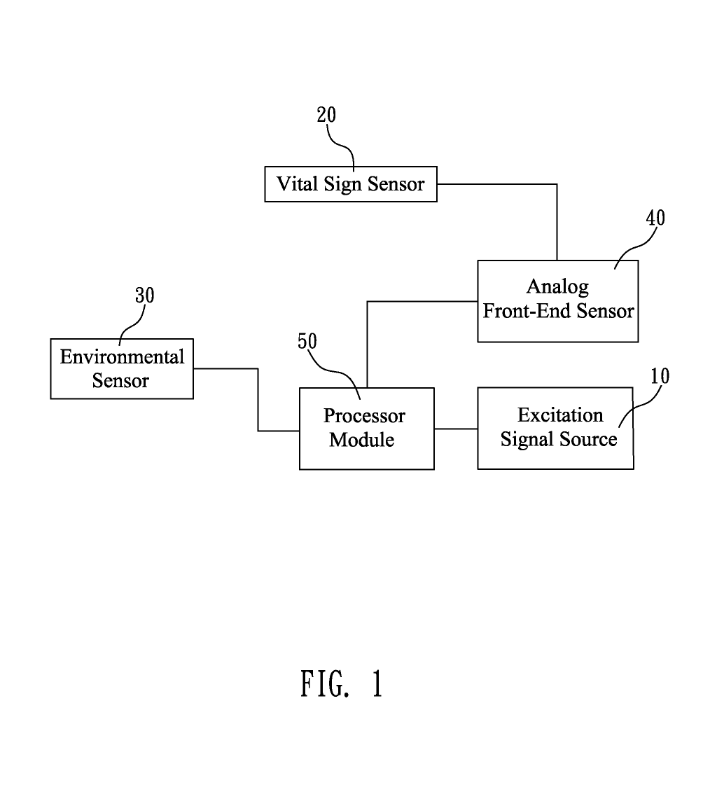

Referring to FIG. 1 to FIG. 5, a vital sign measurement system 100 in accordance with the present invention is shown. The vital sign measurement system 100 adapted for measuring vital signs of a human body (not shown), includes an excitation signal source 10, a vital sign sensor 20, an environmental sensor 30, an analog front-end sensor 40 and a processor module 50.

Referring to FIG. 1 to FIG. 5, the excitation signal source 10 generates excitation signals. The excitation signals are transmitted to the skin of the human body, and then the excitation signals are reflected by the human body to generate the sensing signals. The excitation signals are light signals or biological electrical impulse signals. The light signals are reflected by the skin of the human body to generate light sensing signals. The biological electrical impulse signals are reflected by the skin of the human body to generate biological sensing signals. The sensing signals are the light sensing signals or the biological sensing signals.

Referring to FIG. 1 to FIG. 5, the vital sign sensor 20 receives the sensing signals reflected by the human body. And the sensing signals are converted into a plurality of analog synthetic signals by the vital sign sensor 20. Then the vital sign sensor 20 outputs the analog synthetic signals. Specifically, the vital sign sensor 20 includes at least one of a light sensor 21 for collecting the light sensing signals reflected by the human body, and a bio-impedance sensor 22 for collecting the biological sensing signals. The light sensing signals are converted into the analog synthetic signals by the light sensor 21. The biological sensing signals are converted into the analog synthetic signals by the bio-impedance sensor 22. Then the light sensor 21 outputs the analog synthetic signals. The bio-impedance sensor 22 outputs the analog synthetic signals.

Referring to FIG. 1 to FIG. 5, the environmental sensor 30 receives environmental light of external ambient where the vital sign measurement system 100 is located. The environmental light is a plurality of ultraviolet rays, ambient light or other light. And the environmental light is converted into a plurality of ambient digital indices by the environmental sensor 30. The environmental sensor 30 includes at least one of an ultraviolet sensor 31 and an ambient light sensor 32.

Referring to FIG. 1 to FIG. 5, the analog front-end sensor 40 is electrically connected with the vital sign sensor 20. The analog front-end sensor 40 receives and amplifies the analog synthetic signals of the vital sign sensor 20. And the analog synthetic signals are converted into biological digital signals.

Referring to FIG. 1 to FIG. 5, the processor module 50 is electrically connected with the excitation signal source 10, the environmental sensor 30 and the analog front-end sensor 40. The processor module 50 calculates an intensity of the environmental light of the external ambient where the vital sign measurement system 100 is located on the basis of the ambient digital indices, and the processor module 50 sends an adjustment instruction to the analog front-end sensor 40 to adjust a sampling frequency of the analog front-end sensor 40. The vital sign measurement system 100 adjusts the sampling frequency of the analog front-end sensor 40 according as the environmental sensor 30 senses an intensity change of the environmental light of the external ambient where the vital sign measurement system 100 is located. The analog front-end sensor 40 of which the sampling frequency is adjusted amplifies the analog synthetic signals and converts the analog synthetic signals into the biological digital signals. The processor module 50 proceeds an estimation and a measurement of the vital signs according as the processor module 50 receives the biological digital signals converted by the analog front-end sensor 40 of which the sampling frequency is adjusted.

Referring to FIG. 1 to FIG. 5, specifically, the processor module 50 calculates indices of the ultraviolet rays according to the ultraviolet rays collected by the ultraviolet sensor 31. And the processor module 50 calculates an index of the ambient light according to the ambient light collected by the ambient light sensor 32. When intensities of the ultraviolet rays become larger, the indices of the ultraviolet rays become larger. And when an intensity of the ambient light becomes larger, the index of the ambient light becomes larger.

When the intensity of the environmental light becomes larger, the sampling frequency of the analog front-end sensor 40 is higher. And when the intensity of the environmental light is located becomes smaller, the sampling frequency of the analog front-end sensor 40 is lower, so that measured values of the vital signs affected by the environmental light are balanced.

When the intensity of the environmental light becomes larger, the sampling frequency of the analog front-end sensor 40 sampling the analog synthetic signals of the light sensor 21 is more than 100 Hz. When the intensity of the environmental light becomes smaller, the sampling frequency of the analog front-end sensor 40 sampling the analog synthetic signals of the light sensor 21 is less than 100 Hz. When the intensity of the environmental light becomes larger, the sampling frequency of the analog front-end sensor 40 sampling the analog synthetic signals of the bio-impedance sensor 22 is more than 0.2 Hz. When the intensity of the environmental light becomes smaller, the sampling frequency of the analog front-end sensor 40 sampling the analog synthetic signals of the bio-impedance sensor 22 is less than 0.2 Hz.

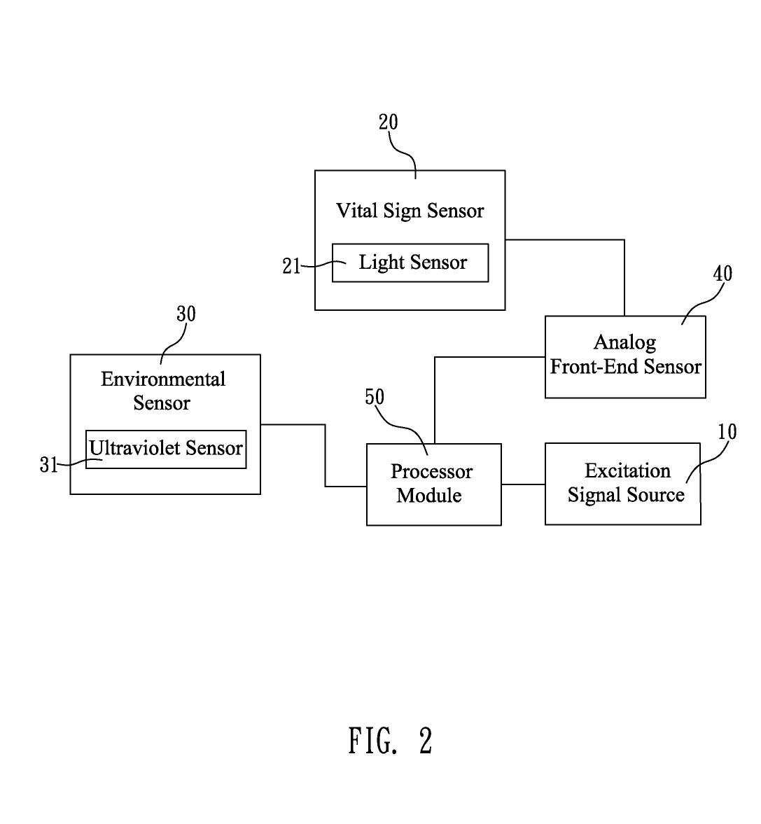

Referring to FIG. 1 and FIG. 2, the vital sign measurement system 100 in accordance with a first embodiment of the present invention is shown. In the first embodiment, the excitation signals are light signals. The sensing signals are light sensing signals. The vital sign sensor 20 includes the light sensor 21. The environmental light is the ultraviolet rays. The light sensor 21 collects the light sensing signals reflected by the human body. The environmental sensor 30 includes the ultraviolet sensor 31. The ultraviolet sensor 31 collects the ultraviolet rays. In the process of adjusting the sampling frequency of the analog front-end sensor 40 sampling the analog synthetic signals of the light sensor 21, when the intensities of the ultraviolet rays become larger, namely the indices of the ultraviolet rays become larger, the sampling frequency of the analog front-end sensor 40 sampling the analog synthetic signals of the light sensor 21 is more than 100 Hz. When the intensities of the ultraviolet rays become smaller, namely the indices of the ultraviolet rays become smaller, the sampling frequency of the analog front-end sensor 40 sampling the analog synthetic signals of the light sensor 21 is less than 100 Hz.

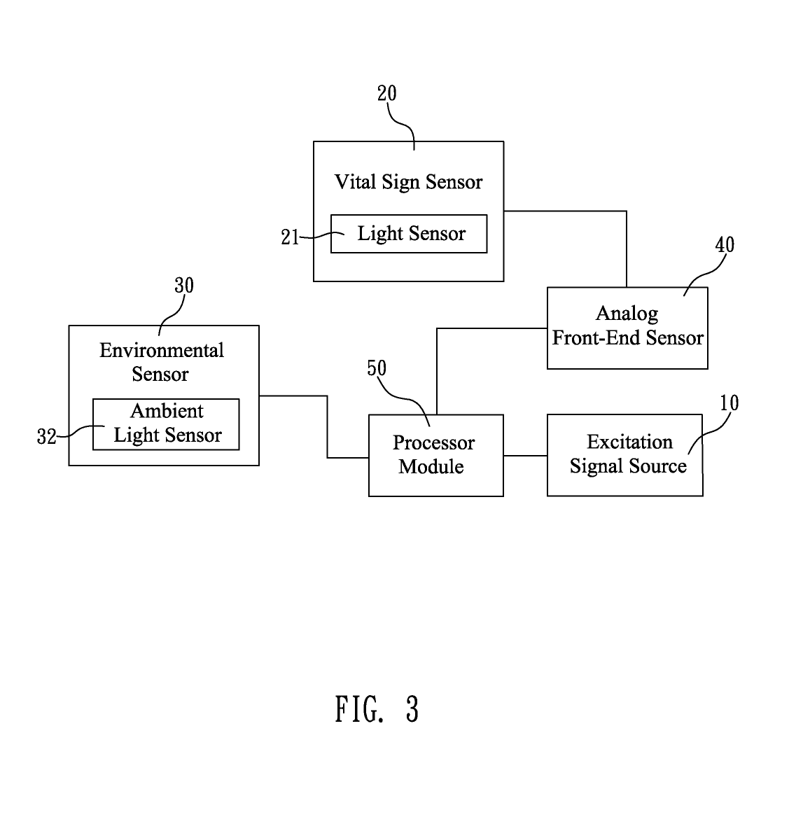

Referring to FIG. 1 and FIG. 3, the vital sign measurement system 100 in accordance with a second embodiment of the present invention is shown. In the second embodiment, the excitation signals are the light signals. The sensing signals are the light sensing signals. The environmental light is the ambient light. The vital sign sensor 20 includes the light sensor 21. The light sensor 21 collects the light sensing signals. The environmental sensor 30 includes the ambient light sensor 32. The ambient light sensor 32 collects the ambient light. In the process of adjusting the sampling frequency of the analog front-end sensor 40 sampling the analog synthetic signals of the light sensor 21, when the intensity of the ambient light becomes larger, namely the index of the ambient light becomes larger, the sampling frequency of the analog front-end sensor 40 sampling the analog synthetic signals of the light sensor 21 is more than 100 Hz. When the intensity of the ambient light becomes smaller, namely the index of the ambient light becomes smaller, the sampling frequency of the analog front-end sensor 40 sampling the analog synthetic signals of the light sensor 21 is less than 100 Hz.

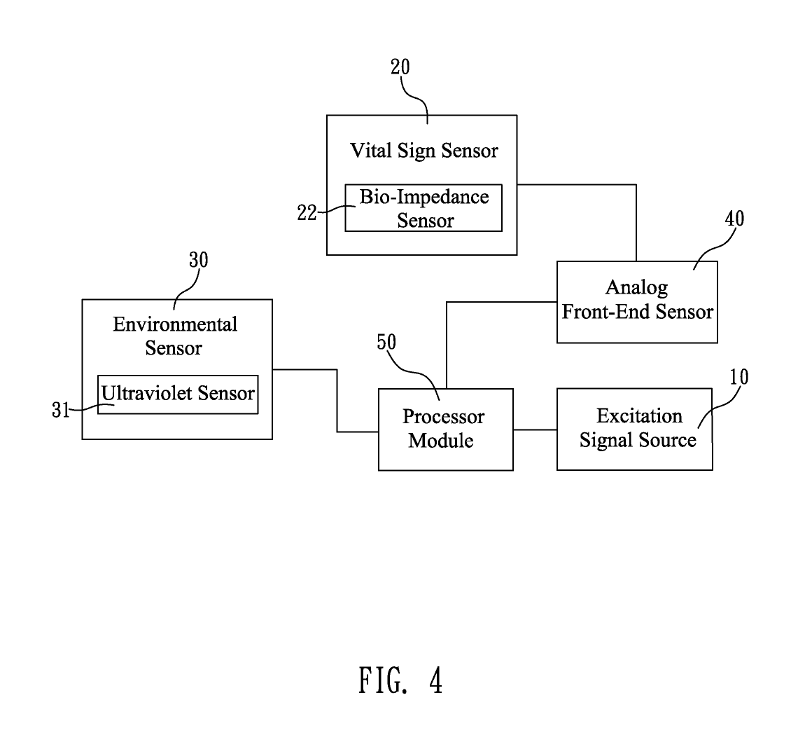

Referring to FIG. 1 and FIG. 4, the vital sign measurement system 100 in accordance with a third embodiment of the present invention is shown. In the third embodiment, the excitation signals are the biological electrical impulse signals. The sensing signals are the biological sensing signals. The environmental light is the ultraviolet rays. The vital sign sensor 20 includes the bio-impedance sensor 22. The bio-impedance sensor 22 collects the biological sensing signals reflected by the human body. The environmental sensor 30 includes the ultraviolet sensor 31. The ultraviolet sensor 31 collects the ultraviolet rays. In the process of adjusting the sampling frequency of the analog front-end sensor 40 sampling the analog synthetic signals of the bio-impedance sensor 22, when the intensities of the ultraviolet rays become larger, namely the indices of the ultraviolet rays become larger, the sampling frequency of the analog front-end sensor 40 sampling the analog synthetic signals of the bio-impedance sensor 22 is more than 0.2 Hz. When the intensities of the ultraviolet rays become smaller, namely the indices of the ultraviolet rays become smaller, the sampling frequency of the analog front-end sensor 40 sampling the analog synthetic signals of the bio-impedance sensor 22 is less than 0.2 Hz.

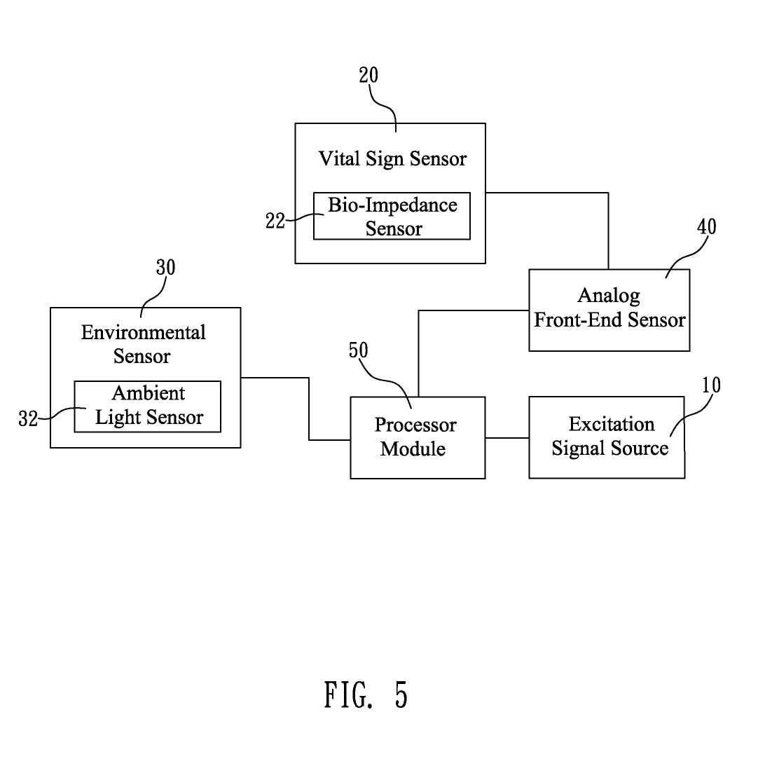

Referring to FIG. 1 and FIG. 5, the vital sign measurement system 100 in accordance with a fourth embodiment of the present invention is shown. In the fourth embodiment, the excitation signals are the biological electrical impulse signals. The sensing signals are the biological sensing signals. The environmental light is the ambient light. The vital sign sensor 20 includes the bio-impedance sensor 22. The bio-impedance sensor 22 collects the biological sensing signals. The environmental sensor 30 includes the ambient light sensor 32. The ambient light sensor 32 collects the ambient light. In the process of adjusting the sampling frequency of the analog front-end sensor 40 sampling the analog synthetic signals of the bio-impedance sensor 22, when the intensity of the ambient light becomes larger, namely the index of the ambient light becomes larger, the sampling frequency of the analog front-end sensor 40 sampling the analog synthetic signals of the bio-impedance sensor 22 is more than 0.2 Hz. When the intensity of the ambient light becomes smaller, namely the index of the ambient light becomes smaller, the sampling frequency of the analog front-end sensor 40 sampling the analog synthetic signals of the bio-impedance sensor 22 is less than 0.2 Hz.

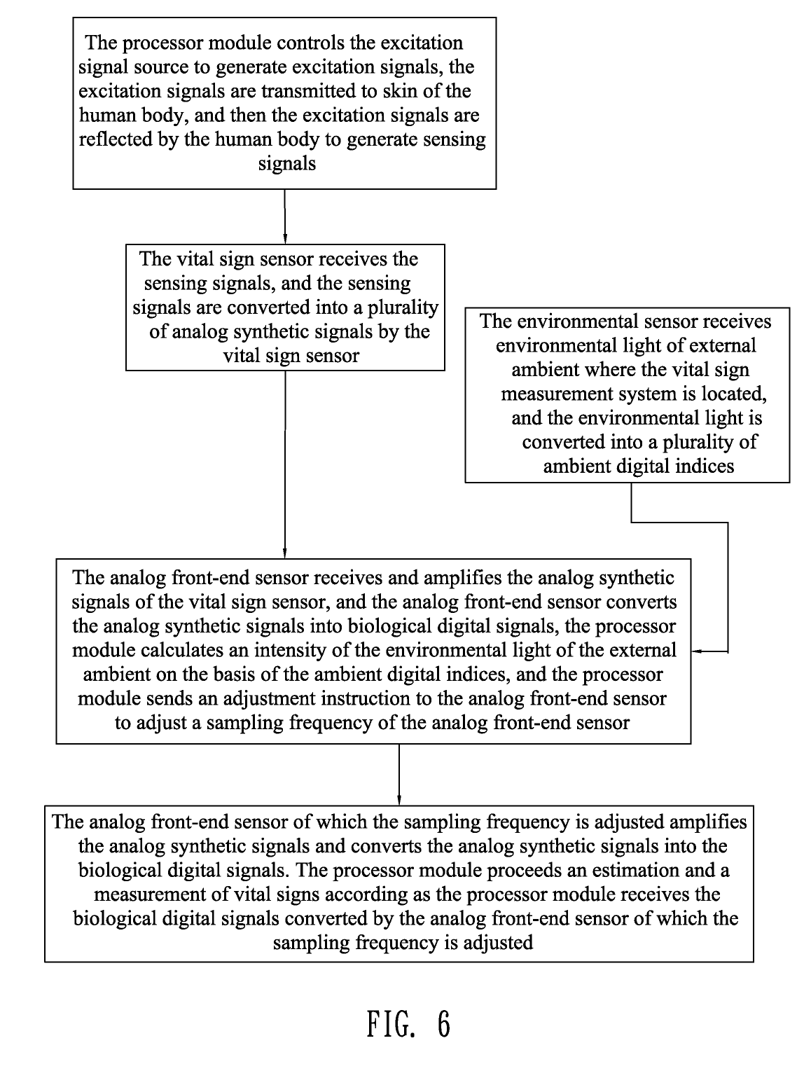

Referring to FIG. 1 to FIG. 6, a vital sign measurement method of the vital sign measurement system 100 includes several steps. Specific steps of vital sign measurement method of the vital sign measurement system 100 are described as follows.

The processor module 50 controls the excitation signal source 10 to generate the excitation signals. The excitation signals are transmitted to the skin of the human body, and then the excitation signals are reflected by the human body to generate the sensing signals.

The vital sign sensor 20 receives the sensing signals, and the sensing signals are converted into a plurality of the analog synthetic signals by the vital sign sensor 20.

The environmental sensor 30 receives the environmental light of external ambient where the vital sign measurement system 100 is located. And the environmental light is converted into a plurality of the ambient digital indices.

The analog front-end sensor 40 receives and amplifies the analog synthetic signals of the vital sign sensor 20. And the analog front-end sensor 40 converts the analog synthetic signals into the biological digital signals. The processor module 50 calculates the intensity of the environmental light of the external ambient on the basis of the ambient digital indices, and the processor module 50 sends the adjustment instruction to the analog front-end sensor 40 to adjust the sampling frequency of the analog front-end sensor 40.

The analog front-end sensor 40 of which the sampling frequency is adjusted amplifies the analog synthetic signals and converts the analog synthetic signals into the biological digital signals. The processor module 50 proceeds the estimation and the measurement of the vital signs according as the processor module 50 receives the biological digital signals converted by the analog front-end sensor 40 of which the sampling frequency is adjusted.

As described above, the vital sign measurement system 100 adjusts the sampling frequency of the analog front-end sensor 40 according as the environmental sensor 30 senses the intensity change of the environmental light of the external ambient where the vital sign measurement system 100 is located, when the intensity of the environmental light becomes larger, the sampling frequency of the analog front-end sensor 40 is higher, when the intensity of the environmental light becomes smaller, the sampling frequency of the analog front-end sensor 40 is lower, so that the measured values of the vital signs affected by the environmental light are balanced. As a result, accuracies of the vital sign measurement system 100 measuring the vital signs are assured.

What is claimed is:

1. A vital sign measurement system, comprising:
an excitation signal source generating excitation signals, the excitation signals being transmitted to skin of a human body, and then the excitation signals being reflected by the human body to generate sensing signals;
a vital sign sensor receiving the sensing signals reflected by the human body, and the sensing signals being converted into a plurality of analog synthetic signals by the vital sign sensor;
an environmental sensor receiving environmental light of external ambient where the vital sign measurement system is located, and the environmental light being converted into a plurality of ambient digital indices by the environmental sensor;
an analog front-end sensor electrically connected with the vital sign sensor, the analog front-end sensor receiving and amplifying the analog synthetic signals of the vital sign sensor, and the analog synthetic signals being converted into biological digital signals; and
a processor module electrically connected with the excitation signal source, the environmental sensor and the analog front-end sensor, the processor module calculating an intensity of the environmental light of the external ambient on the basis of the ambient digital indices, and the processor module sending an adjustment instruction to the analog front-end sensor to adjust a sampling frequency of the analog front-end sensor according as the environmental sensor senses an intensity change of the environmental light of the external ambient, the processor module proceeding an estimation and a measurement of vital signs according as the processor module receives the biological digital signals converted by the analog front-end sensor of which the sampling frequency is adjusted.

2. The vital sign measurement system as claimed in claim 1, wherein the excitation signals are light signals, the sensing signals are light sensing signals, the vital sign sensor includes a light sensor, the light sensor collects the light sensing signals reflected by the human body.

3. The vital sign measurement system as claimed in claim 2, wherein when the intensity of the environmental light becomes larger, the sampling frequency of the analog front-end sensor sampling the analog synthetic signals of the light sensor is more than 100 Hz, when the intensity of the environmental light becomes smaller, the sampling frequency of the analog front-end sensor sampling the analog synthetic signals of the light sensor is less than 100 Hz.

4. The vital sign measurement system as claimed in claim 3, wherein the environmental sensor includes an ultraviolet sensor, the environmental light of the external ambient is ultraviolet rays, the ultraviolet sensor collects the ultraviolet rays, when intensities of the ultraviolet rays become larger, the sampling frequency of the analog front-end sensor sampling the analog synthetic signals of the light sensor is more than 100 Hz, when the intensities of the ultraviolet rays become smaller, the sampling frequency of the analog front-end sensor sampling the analog synthetic signals of the light sensor is less than 100 Hz.

5. The vital sign measurement system as claimed in claim 3, wherein the environmental sensor includes an ambient light sensor, the environmental light of the external ambient is ambient light, the ambient light sensor collects the ambient light, when an intensity of the ambient light becomes larger, the sampling frequency of the analog front-end sensor sampling the analog synthetic signals of the light sensor is more than 100 Hz, when the intensity of the ambient light becomes smaller, the sampling frequency of the analog front-end sensor sampling the analog synthetic signals of the light sensor is less than 100 Hz.

6. The vital sign measurement system as claimed in claim 1, wherein the excitation signals are biological electrical impulse signals, the sensing signals are biological sensing signals, the vital sign sensor includes a bio-impedance sensor, the bio-impedance sensor collects the biological sensing signals reflected by the human body.

7. The vital sign measurement system as claimed in claim 6, wherein when the intensity of the environmental light becomes larger, the sampling frequency of the analog front-end sensor sampling the analog synthetic signals of bio-impedance sensor is more than 0.2 Hz, when the intensity of the environmental light becomes smaller, the sampling frequency of the analog front-end sensor sampling the analog synthetic signals of bio-impedance sensor is less than 0.2 Hz.

8. The vital sign measurement system as claimed in claim 7, wherein the environmental sensor includes an ultraviolet sensor, the environmental light of the external ambient is ultraviolet rays, the ultraviolet sensor collects the ultraviolet rays, when intensities of the ultraviolet rays become larger, the sampling frequency of the analog front-end sensor sampling the analog synthetic signals of the bio-impedance sensor is more than 0.2 Hz, when the intensities of the ultraviolet rays become smaller, the sampling frequency of the analog front-end sensor sampling the analog synthetic signals of the bio-impedance sensor is less than 0.2 Hz.

9. The vital sign measurement system as claimed in claim 7, wherein the environmental sensor includes an ambient light sensor, the environmental light of the external ambient is ambient light, the ambient light sensor collects the ambient light, when an intensity of the ambient light becomes larger, the sampling frequency of the analog front-end sensor sampling the analog synthetic signals of the bio-impedance sensor is more than 0.2 Hz, when the intensity of the ambient light becomes smaller, the sampling frequency of the analog front-end sensor sampling the analog synthetic signals of the bio-impedance sensor is less than 0.2 Hz.

10. A vital sign measurement method of the vital sign measurement system which includes an excitation signal source, a vital sign sensor, an environmental sensor, an analog front-end sensor and a processor module, comprising the steps of:
the processor module controlling the excitation signal source to generate excitation signals, the excitation signals being transmitted to skin of the human body, and then the excitation signals being reflected by the human body to generate sensing signals;
the vital sign sensor receiving the sensing signals, and the sensing signals being converted into a plurality of analog synthetic signals by the vital sign sensor;
the environmental sensor receiving environmental light of external ambient where the vital sign measurement system is located, and the environmental light being converted into a plurality of ambient digital indices;
the analog front-end sensor receiving and amplifying the analog synthetic signals of the vital sign sensor, and the analog front-end sensor converting the analog synthetic signals into biological digital signals, the processor module calculating an intensity of the environmental light of the external ambient on the basis of the ambient digital indices, and the processor module sending an adjustment instruction to the analog front-end sensor to adjust a sampling frequency of the analog front-end sensor; and
the analog front-end sensor of which the sampling frequency is adjusted amplifying the analog synthetic signals and converting the analog synthetic signals into the biological digital signals, the processor module proceeding an estimation and a measurement of vital signs according as the processor module receives the biological digital signals converted by the analog front-end sensor of which the sampling frequency is adjusted.
